(19) United States
(12) Patent Application Publication
(10) Pub. No.: US 2025/0082912 A1
(43) Pub. Date: Mar. 13, 2025

(54) BALLOON CATHETER METHODS OF USE

(71) Applicant: HOPE MEDICAL, Cincinnati, OH (US)

(72) Inventors: Andrew GEORGILIS, Cincinnati, OH (US); Paul IMAOKA, Grayslake, IL (US); Brandon VERWIJST, Grayslake, IL (US)

(21) Appl. No.: 18/464,859

(22) PCT Filed: Mar. 14, 2022
(86) PCT No.: PCT/US22/20152

§ 371 Date: Sep. 11, 2023

Related U.S. Application Data
(60) Provisional application No. 63/160,477, filed on Mar. 12, 2021.

(51) Int. Cl. A61M 25/10; A61M 25/00

(52) U.S. Cl. CPC A61M 25/1018; A61M 25/0009

(57) ABSTRACT

A method of using a balloon catheter is disclosed. One embodiment of this method may include coaxially disposing an inner catheter through an outer catheter and through a distal balloon, operably coupling a proximal hub with a distal balloon through the outer catheter and the inner catheter, maintaining an inner lumen in the inner catheter to pass over a guidewire, maintaining the outer catheter with a fluid pathway channel by which the distal balloon is inflated from a contracted state, including a distal end on the inner catheter in which a distal tip is operably disposed, and inflating the distal balloon into a unique shape using the fluid pathway channel of the outer catheter.

### CROSS REFERENCE TO RELATED APPLICATIONS

The present disclosure claims the benefit of U.S. Provisional Application No. 63/160,477 filed on Mar. 12, 2021, the contents of which are hereby incorporated herein by reference in entirety.

### FIELD OF DISCLOSURE

The invention generally relates to catheters, and more specifically, catheters for esophageal dilation.

### BACKGROUND

Upper esophageal sphincter (UES) dysfunction occurs when the muscles of the upper esophagus, specifically the cricopharyngeus, develop impaired relaxation. Symptoms include progressive dysphagia from pills to solids and then liquids. Diagnosis is made from a thorough history and Modified Barium Swallow Study (MBSS).

Current esophageal dilators are cylindrical and have been developed to dilate the esophageal body that has a round circumference or the upper esophageal sphincter (UES). Causes of UES dysfunction include acid reflux disease, cricopharyngeus muscle dysfunction, and upper esophageal web. To treat this affliction, an esophageal dilator can be dilated within the UES for a period of time to help remodel the scar tissue.

The present invention solves these problems, as well as others.

### SUMMARY OF THE INVENTION

Provided herein are systems and methods for a balloon dilation catheter. One method of using a balloon catheter includes coaxially disposing an inner catheter through an outer catheter and through a distal balloon; operably coupling a proximal hub with the distal balloon through the outer catheter and the inner catheter; maintaining an inner lumen in the inner catheter to pass over a guidewire; maintaining the outer catheter with a fluid pathway channel by which the distal balloon is inflated from a contracted state; including a distal end on the inner catheter in which a distal tip is operably disposed; and inflating the distal balloon into a unique shape using the fluid pathway channel of the outer catheter.

In one embodiment, the method further includes using a radiopaque marker on the outer catheter for visualization of the balloon catheter when operably disposed in a patient. In a second embodiment, the unique shape of the balloon further comprises: a first balloon profile with a proximal conical section tapered to join the proximal portion, a proximal expanded ring connected to the proximal conical section, a proximal tapered portion connected to the proximal expanded ring, a central elongated portion connected to the proximal tapered portion, a distal tapered portion connected to the central elongated portion, a distal expanded ring connected to the distal tapered portion, and a distal conical section connected to the distal expanded ring; and a second balloon profile with a proximal conical section tapered to join the proximal portion, a proximal expanded ring connected to the proximal conical section, a proximal tapered portion connected to the proximal expanded ring, a central elongated portion connected to the proximal tapered portion, a distal tapered portion connected to the central elongated portion, a distal expanded ring connected to the distal tapered portion, and a distal conical section connected to the distal expanded ring.

In some embodiments, the method of using a catheter for dilating an upper esophageal sphincter includes providing a substantially deflated balloon; coupling the balloon a hub through an outer catheter and an inner catheter wherein the outer catheter is coaxially coupled to the inner catheter; positioning the balloon in a desired location; inflating the balloon to a pressure that is below the burst pressure of the balloon; deflating the balloon; and removing the balloon from the desired location.

In one example of this embodiment, the desired location for positioning and removing the balloon is located at least partially within an upper esophageal sphincter. In one embodiment, the balloon at least partially dilates the upper esophageal sphincter when the balloon is inflated. In some embodiments, the balloon is comprised of a semi-compliant material selected from the group consisting of polyamides, and engineered nylons such as Pebax®, Grilamid®, and Vestamid®, PET, and high durometer polyurethane. The burst pressure of the balloon is between about 15 psi to about 265 psi. In yet another embodiment of this disclosure, inflating the balloon further includes a first stage of expansion, a second stage of expansion, and a third stage of expansion; wherein the diameter of the balloon in the second stage of expansion is larger than the diameter of the balloon in the first stage of expansion; further wherein the diameter of the balloon in the third stage of expansion is larger than the diameter of the balloon in the second stage of expansion. In another example, the diameter of the balloon in the first stage is not less than 16 mm and not greater than 38 mm;

Another embodiment of the present disclosure includes a balloon catheter kit, which comprises a balloon assembly; a proximal hub; an outer catheter tube; an inner catheter which is coaxially couplable to the outer catheter tube; a distal tip which is operably couplable to the inner catheter at a distal end; a container configured to removably contain the balloon assembly, proximal hub, outer catheter tube, inner catheter tube, and distal tip, among other items; wherein the balloon assembly is couplable with the proximal hub through the outer catheter tube and the inner catheter; further wherein the outer catheter maintains a fluid pathway channel by which the balloon is inflated; and wherein the inner catheter maintains an inner lumen to pass over a guidewire.

In some embodiments, the balloon assembly further includes a first balloon profile with a proximal conical section tapered to join the proximal portion, a proximal expanded ring connected to the proximal conical section, a proximal tapered portion connected to the proximal expanded ring, a central elongated portion connected to the proximal tapered portion, a distal tapered portion connected to the central elongated portion, a distal expanded ring connected to the distal tapered portion, and a distal conical section connected to the distal expanded ring; and a second balloon profile with a proximal conical section tapered to join the proximal portion, a proximal expanded ring connected to the proximal conical section, a proximal tapered portion connected to the proximal expanded ring, a central elongated portion connected to the proximal tapered portion, a distal tapered portion connected to the central elongated portion, a distal expanded ring connected to the distal tapered portion, and a distal conical section connected to the distal expanded ring.

In one embodiment, the balloon assembly also includes a first balloon, second balloon, and third balloon; wherein the second balloon has a cross-section that is larger than the cross-section of the first balloon and smaller than the cross-section of the third balloon when the three balloons are inflated. In some embodiments, each of the three balloons are kidney shaped when inflated.

### BRIEF DESCRIPTION OF THE DRAWINGS

In the accompanying figures, like elements are identified by like reference numerals among the several preferred embodiments of the present invention.

FIG. 1A is a perspective view of the balloon catheter apparatus.

FIG. 1B is an exploded view of the balloon catheter apparatus.

FIG. 2A is a perspective view of the distal balloon.

FIG. 2B is a cross-sectional view of the distal balloon.

FIG. 2C is a top view of the distal balloon.

FIG. 2D is a distal view of the distal balloon.

FIG. 3 is a cross-sectional view of the distal balloon coupled with the inner catheter and the outer catheter.

FIG. 4 is a cross-sectional view of the hub.

FIG. 5 is a schematic of the esophagus and the upper esophageal sphincter (UES) to be dilated by the balloon catheter.

FIG. 6 is a schematic representation of a kit containing embodiments of the balloon dilation catheter discussed herein.

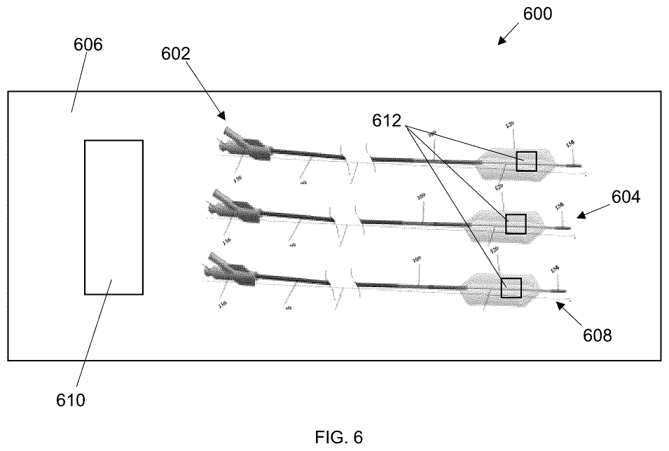

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising.” “includes,” and/or “including.” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

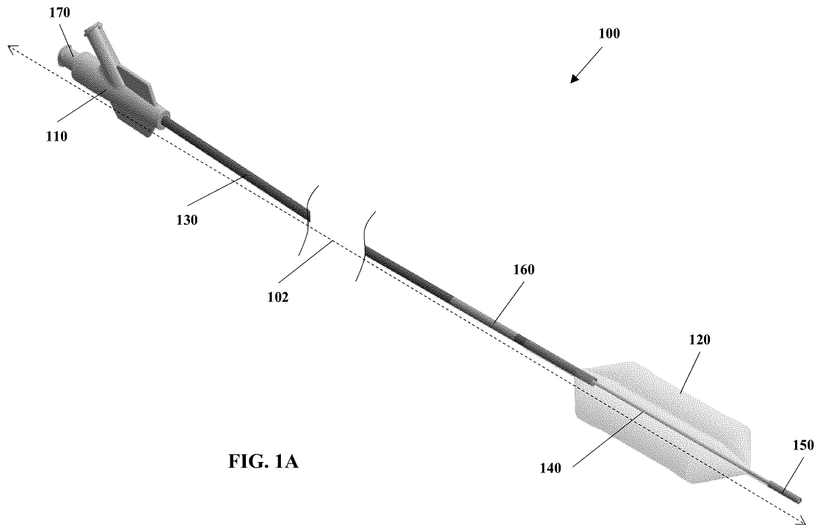

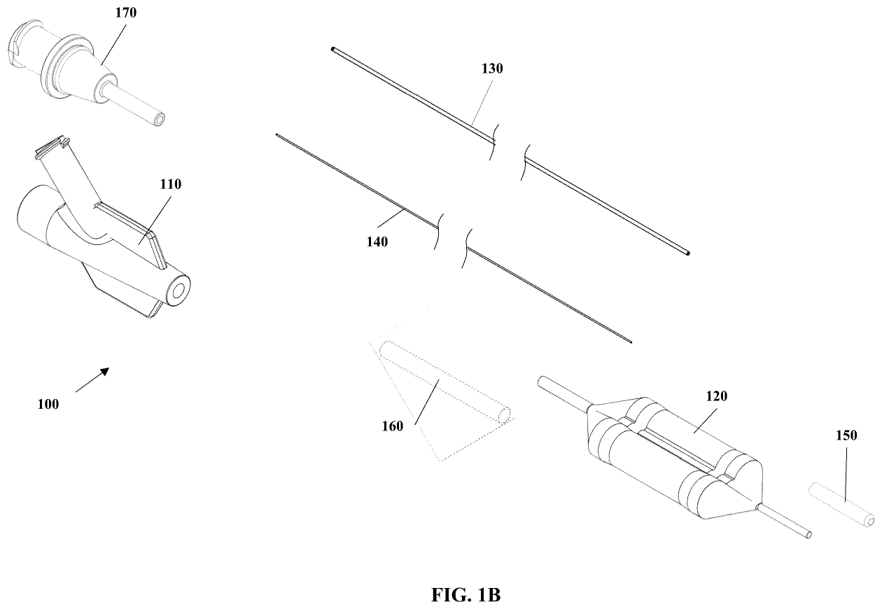

Generally speaking, the Balloon Catheter Apparatus travels over a guidewire and dilates the upper esophageal sphincter using a semi-compliant, single balloon with a shape conducive to the anatomy of the upper esophageal sphincter. As shown in FIGS. 1A-1B, the balloon catheter 100 comprises a proximal hub 110 operably coupled with a distal balloon 120 through an outer catheter tube 130 and an inner catheter 140 generally along a longitudinal axis 102 of the balloon catheter 100. The outer catheter 130 is coaxially coupled with the inner catheter 140. The inner catheter 140 maintains an inner lumen to pass over the guidewire. The outer catheter 130 maintains a fluid pathway channel by which the distal balloon 120 is inflated from a contracted or deflated state. The inner catheter 140 coaxially runs through both the outer catheter 130 and the distal balloon 120. The inner catheter 140 may include a distal end in which a distal tip 150 is operably disposed. The outer catheter 130, inner catheter 140, or distal balloon 120 may include a radiopaque marker 160 for visualization of the balloon catheter 100 when operably disposed in a patient. The proximal hub 110 may include an inner hub portion 170 that is operably coupled with the inner catheter 140, while the proximal hub 110 is operably coupled with the outer catheter 130.

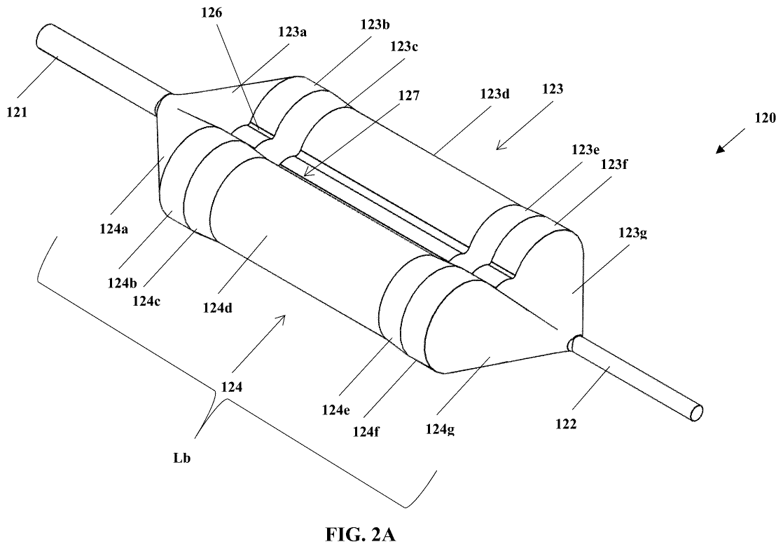

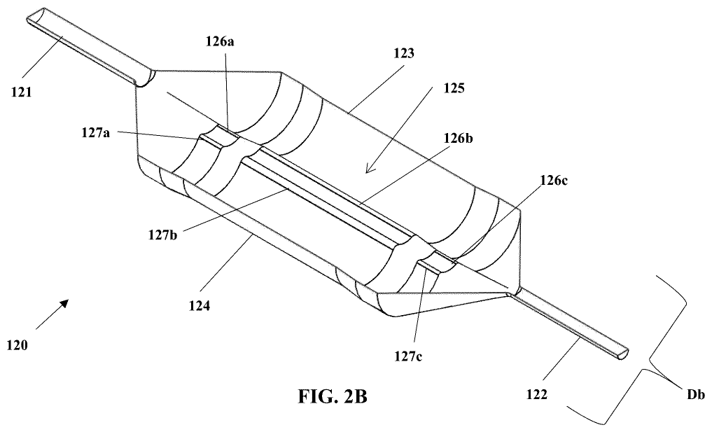

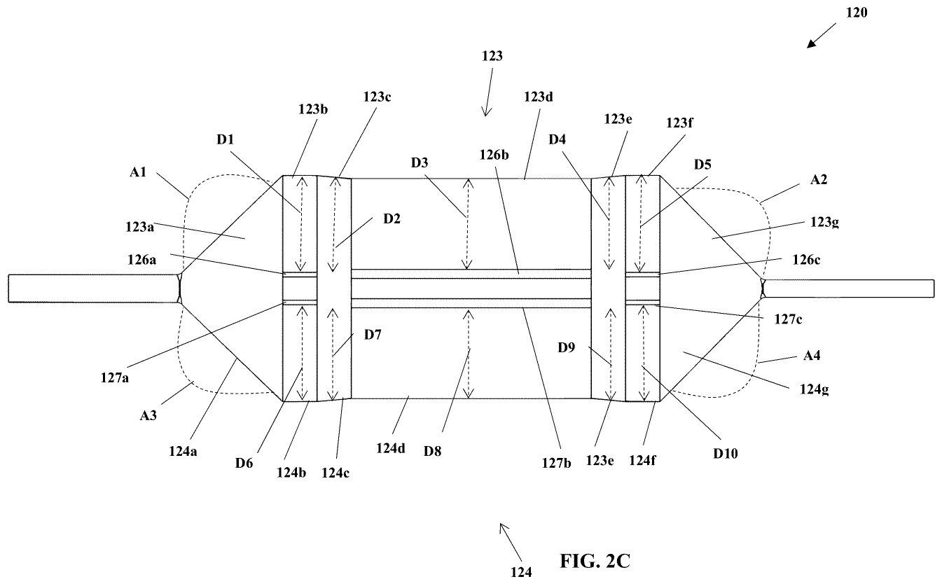

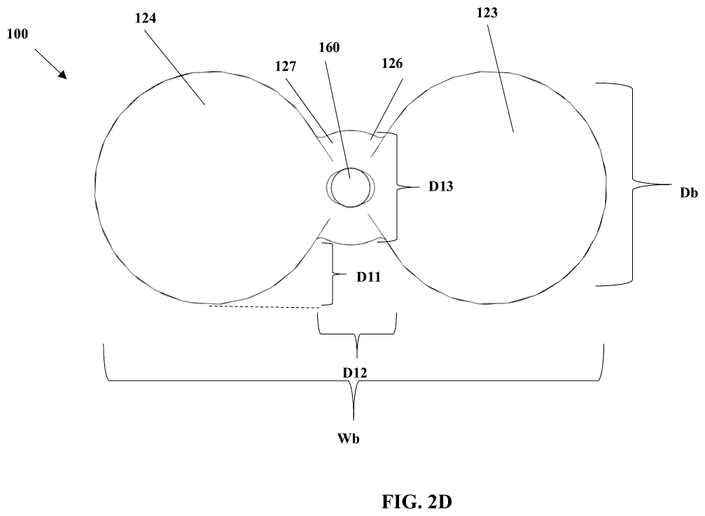

As shown in FIGS. 2A-2C, the distal balloon 120 may include a proximal portion 121 and a distal portion 122 generally running along the longitudinal axis. The distal balloon 120 may include a length Lb, a Width Wb, and a diameter Db generally approximating the length, width, and diameter of the upper esophageal sphincter. The distance around the width Wb and the diameter Db from a cross-sectional perspective may be considered the circumference of the distal balloon 120. The distal portion 122 is operably coupled with the distal tip 150 and set to receive the inner catheter 140. The proximal portion 121 is coaxially coupled with the outer catheter 130. The distal balloon 120 may include a first balloon 123 and a second balloon 124 operably coupled with the proximal portion 121 and the distal portion 122. As shown in FIG. 2B, the first balloon 123 and the second balloon 124 include a balloon lumen 125 to receive the fluid delivered through the fluid passageway disposed between the outer catheter 130 and the inner catheter 140. The first balloon 123 may include a first longitudinal depression 126 and the second balloon 124 may include a second longitudinal depression 127, wherein the first longitudinal depression 126 and second longitudinal depression 127 are disposed between the first balloon 123 and the second balloon 124. The first balloon 123 may include a first balloon profile that may be generally similar to a second balloon profile of the second balloon 124, wherein the first balloon profile and the second balloon profile approximates the shape of the upper esophageal sphincter when the balloon profile is in the expanded or inflated state. The first balloon and the second balloon is substantially monolithic and may include a single lumen in which the distal balloon is expanded from the contracted, or deflated, state to the expanded, or inflated, state. In one embodiment, the distal balloon may include a stage 1 expanded state, a stage 2 expanded state. In another embodiment, the distal balloon 120 may also include a stage 3 expanded state, as further detailed in the Table 1 below. However, fewer or more expanded states of the distal balloon 120 are also contemplated herein.

In one embodiment, diameter Db is less than 75% of the Width Wb, which approximates the shape of the upper esophageal sphincter. In one embodiment, the length Lb is greater than about 35 mm. In one embodiment, the diameter of the inner catheter is about 2.50 millimeters. In one embodiment, the diameter of the outer catheter is about 4.00 millimeters.

As shown in FIGS. 2A-2C, the first balloon profile comprises a proximal conical section 123a tapered to join the proximal portion 121, a proximal expanded ring 123b connected to the proximal conical section 123a, a proximal tapered portion 123c connected to the proximal expanded ring 123b, a central elongated portion 123d connected to the proximal tapered portion 123c, a distal tapered portion 123e connected to the central elongated portion 123d, a distal expanded ring 123f connected to the distal tapered portion 123e, and a distal conical section 123g connected to the distal expanded ring 123f. The second balloon profile comprises a proximal conical section 124a tapered to join the proximal portion 121, a proximal expanded ring 124b connected to the proximal conical section 124a, a proximal tapered portion 124c connected to the proximal expanded ring 124b, a central elongated portion 124d connected to the proximal tapered portion 124c, a distal tapered portion 124e connected to the central elongated portion 124d, a distal expanded ring 124f connected to the distal tapered portion 124e, and a distal conical section 124g connected to the distal expanded ring 124f.

The first longitudinal depression 126 may include a proximal longitudinal depression 126a operably connected to the proximal expanded ring 123b, a central elongated depression 126b operably connected to the central elongated portion 123d, and a distal longitudinal depression 126c operably connected to the distal expanded ring 123f. The second longitudinal depression 127 may include a proximal longitudinal depression 127a operably connected to the proximal expanded ring 124b, a central elongated depression 127b operably connected to the central elongated portion 124d, and a distal longitudinal depression 127c operably connected to the distal expanded ring 124f.

The proximal conical section 123a may include an angle A1 in which the proximal conical section 123a connects to the proximal portion 121. The proximal expanded ring 123b may include a diameter D1, the proximal tapered portion 123c may include a diameter D2, the central elongated portion 123d may include a diameter D3, the distal tapered portion 123e may include a diameter D4, the distal expanded ring 123f may include a diameter D5, and the distal conical section 123g may include an angle A2 in which the distal conical section 123g connects to the distal portion 122. The proximal conical section 124a may include an angle A3 in which the proximal conical section 124a connects to the proximal portion 121. The proximal expanded ring 124b may include a diameter D6, the proximal tapered portion 124c may include a diameter D7, the central elongated portion 124d may include a diameter D8, the distal tapered portion 124e may include a diameter D9, the distal expanded ring 124f may include a diameter D10, and the distal conical section 124g may include an angle A4 in which the distal conical section 124g connects to the distal portion 122.

In one embodiment, the angle A1-A4 is between about 20-60 degrees, between about 30-50 degrees, between about 40-49 degrees. In one embodiment, the Diameters D1-D10 is between about 1 mm and about 15 mm, between about 5 mm and about 10 mm, between about 7 mm and about 12 mm.

The proximal longitudinal depression 126a maintains diameter D1 of the proximal expanded ring 123b in the expanded state, the central elongated depression 126b maintains the diameter D3 of the central elongated portion 123d in the expanded state, and the distal longitudinal depression 126c maintains the diameter D5 the distal expanded ring 123f in the expanded state. The proximal longitudinal depression 127a maintains the diameter D6 of the proximal expanded ring 124b, the central elongated depression 127b maintains the diameter D8 of the central elongated portion 124d in the expanded state, and the distal longitudinal depression 127c maintains the diameter D10 of the distal expanded ring 124f in the expanded state. The proximal tapered portion 123c and the proximal tapered portion 124b do not include a longitudinal depression, in one embodiment, and the proximal tapered portion 123c and the proximal tapered portion 124b may include a longitudinal depression. The distal tapered portion 123e and the distal tapered portion 124e do not include a longitudinal depression, in one embodiment, the distal tapered portion 123e and the distal tapered portion 124e may include a longitudinal depression in other embodiments. The first longitudinal depression 126 and the second longitudinal depression 127 maintain a separation distance D11 between the diameter Db of the balloon catheter, a separation width D12 between the first balloon 123 and the second balloon 124, and a diameter D13, as shown in FIG. 2D. The separation distance D11, the diameter Db, and the diameter D13 are formulated to estimate the upper esophageal sphincter when the balloon catheter 100 is in the expanded state. The diameter D3 of the first longitudinal depression 126 and the second longitudinal depression 127 maintain the first balloon and the second balloon in the expanded state in multiple expansion states. The diameter D11 is between about 1 mm and about 15 mm, between about 5 mm and 10 mm, or between about 7 mm and about 9 mm. The diameter D12 is between about 1 mm and about 15 mm, between about 5 mm and 10 mm, or between about 7 mm and about 9 mm. The diameter D13 is between about 1 mm and about 17 mm, between about 5 mm and 15 mm, or between about 7 mm and about 12 mm.

According to one embodiment, the diameter D1 of the proximal expanded ring 123b is greater than the diameter D2 of the proximal tapered portion 123c. The diameter D3 of the central elongated portion 123d is less than the diameter D2 of the proximal tapered portion 123c. The diameter D4 of the distal tapered portion 123e is greater than the diameter D3 of the central elongated portion 123d. The diameter D5 of the distal expanded ring 123f is greater than the diameter D4 of the distal tapered portion 123c. The diameter D6 of the proximal expanded ring 124b is greater than the diameter D7 of the proximal tapered portion 124c. The diameter D8 of the central elongated portion 124d is less than the diameter D7 of the proximal tapered portion 124c. The diameter D9 of the distal tapered portion 124e is greater than the diameter D8 of the central elongated portion 124d. The diameter D10 of the distal expanded ring 124f is greater than the diameter D9 of the distal tapered portion 124e. The diameters D1-D10 are formulated to estimate the upper esophageal sphincter and dilate the same.

The diameters D1-D10 may vary for differently sized distal balloons 120 as discussed herein. For example, in a kit embodiment considered herein, some or all of the diameters D1-D10 may vary among two or more distal balloons 120 of the balloon dilation catheters 100 provided in the kit. For example, some or all of the diameters D1-D10 may increase between a first dilation catheter provided in the kit and a second balloon dilation catheter in the kit. Further, a third balloon dilation catheter may be provided in the kit having a distal balloon 120 with one or more diameter D1-D10 that are great than the first and second balloon dilation catheters provided in the kit. Such a kit may allow a medical professional to select which balloon dilation catheter is sized to address the particular anatomy of the patient for which it is to be used. Further still, the medical professional may progressively use balloon dilation catheters within the kit to transition from a balloon dilation catheter with one or more smaller diameter D1-D10 to a balloon dilation catheter from the kit with one or more comparatively larger diameters D1-D10 to sequentially expand the larger diameter balloon dilation catheter in the UES to remedy the constriction.

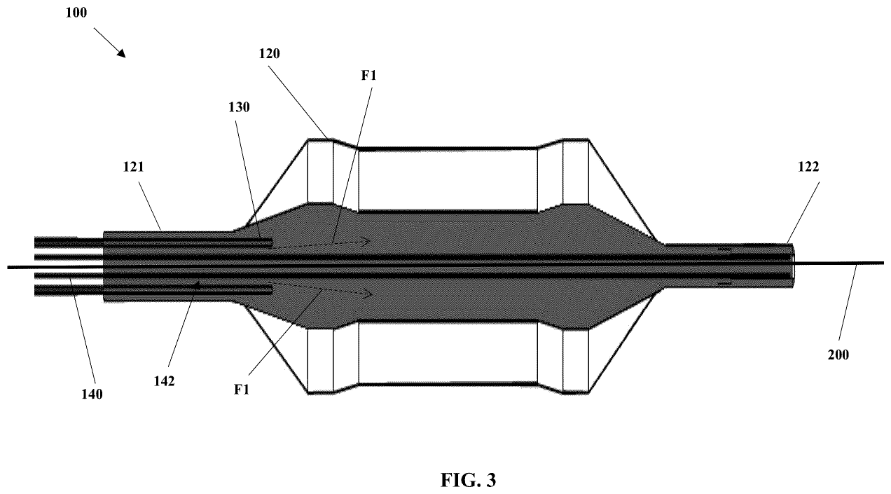

As shown in the FIG. 3, the distal balloon 120 is operably coupled with the inner catheter 140 and the outer catheter 130. The proximal portion 121 of the distal balloon 120 forms a seal with the outer catheter 130, while the outer catheter 130 forms a fluid pathway 142 between the inner catheter 140. In operation, a fluid F1 is disposed through the proximal hub 110 and travels distally toward the distal balloon 120 and the fluid F1 inflates the distal balloon 120 to the expanded state. The distal balloon 120 forms a seal with the inner catheter 140 and the distal portion 122 of the distal balloon.

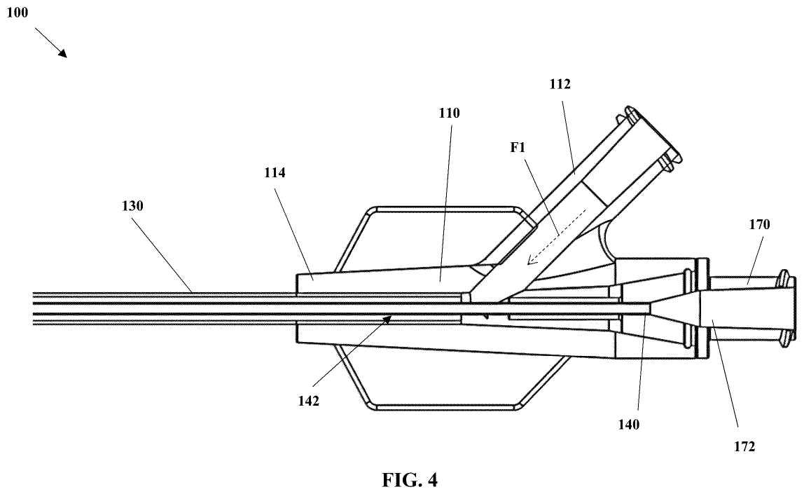

As shown in FIG. 4, the proximal hub 110 may include the inner hub portion 170 that is operably coupled with the inner catheter 140. The proximal hub 110 may include a fluid lumen 112 that operably couples with a fluid source (not shown) to deliver a fluid F1 distally from the proximal hub 110 to the fluid pathway 142. The fluid pathway 142 is created coaxially between the outer surface of the inner catheter 140 and the inner surface of the outer catheter 130. The outer catheter is sealed to the distal portion 114 of the proximal hub 110 to form the fluid pathway 142 from the fluid lumen 112. The inner catheter 140 is operably coupled with a lumen 172 of the inner hub portion 170 that allows for a guidewire 200 to be disposed coaxially within the inner catheter 140 for proper placement of the distal balloon 120, as shown in FIG. 3.

According to some embodiments, a method of using a balloon catheter may include coaxially disposing an inner catheter 140 through an outer catheter 130 and through a distal balloon 120. Then, a proximal hub 110 may be operably coupled with the distal balloon 120 through the outer catheter 130 and the inner catheter 140. In other words, both the outer catheter 130 and the inner catheter 140 may be coupled to the proximal hub 110 at a proximal end, and may be coupled to a balloon 120 at a distal end. The inner lumen in the inner catheter may be maintained so the inner catheter may pass over a guidewire. The guidewire may direct the inner catheter 140 and the components coupled thereto—such as the balloon 120 and the outer catheter 130—to a desired location. The outer catheter 130 may maintain a fluid pathway channel. The distal balloon 120 may be inflated from a contracted state through this fluid pathway channel. The next step may be including a distal end on the inner catheter in which a distal tip 150 is operably disposed. Then, the method may include inflating the distal balloon 120 into a unique shape using the fluid pathway channel of the outer catheter 130. Inflating the balloon 120 may dilate the upper esophageal sphincter. This method may also comprise of a radiopaque marker 160 located on the outer catheter 130. This may help with visualization of the balloon 120 catheter when it is operably disposed, such as in a patient.

In another embodiment, the method of using a catheter to dilate an upper esophageal sphincter may include the steps of (1) providing a substantially deflated balloon; (2) coupling the balloon to the hub through an outer catheter and an inner catheter, wherein the outer catheter is coaxially coupled to the inner catheter; (3) positioning the balloon in a desired location; (4) inflating the balloon to a pressure that is below the burst pressure of the balloon; (5) deflating the balloon; and (6) removing the balloon from the desired location. While the desired location may be the upper esophageal sphincter, one of ordinary skill in the art would understand that this may be used in other areas of a body.

The unique shape of the balloon 120, mentioned above, may be the shape of a kidney in one embodiment. The shape of the balloon 120 might also include a first balloon profile with a proximal conical section tapered to join the proximal portion, a proximal expanded ring connected to the proximal conical section, a proximal tapered portion connected to the proximal expanded ring, a central elongated portion connected to the proximal tapered portion, a distal tapered portion connected to the central elongated portion, a distal expanded ring connected to the distal tapered portion, and a distal conical section connected to the distal expanded ring. There additionally may be a second balloon profile that substantially mirrors the profile of the first balloon profile.

### Semi-Compliant Balloon Properties

The distal balloon is preferably made from a semi-compliant material, including, but not limited to: polyamides and engineered nylons such as Pebax®, Grilamid®, and Vestamid®, PET, and high durometer polyurethane. The distal balloon includes a compliance range between about 5% to about 15%. The distal balloon includes a burst pressure between about 15 psi to about 375 psi (˜1 to ˜25 atm). The distal balloon includes a rated burst pressure (RBP) between about 75 psi to about 265 psi (˜5 to ˜18 atm). In one embodiment, the distal balloon withstands at least 10 cycles of expansion to the expanded state from the contracted state. In one embodiment, the distal balloon holds for at least 30 seconds in the expanded state to sufficiently dilate the esophageal sphincter. In one embodiment, the distal balloon may include a first stage of expansion, a second stage of expansion, and a third stage of expansion. The first, second, and third stages of expansion may be according to Table 1 below.

TABLE 1

| | Stage 1 | Stage 2 | Stage 3 | Burst |
|---|---|---|---|---|
| Diameter | (mm) | (mm) | (mm) | pressure |
| 17 | 15 | 16 | 17 | >120% |
| | | | | Stage 3 |
| 20 | 18 | 19 | 20 | >120% |
| | | | | Stage 3 |
| 23 | 21 | 22 | 23 | >120% |
| | | | | Stage 3 |
| 26 | 24 | 25 | 26 | >120% |
| | | | | Stage 3 |
| 29 | 27 | 28 | 29 | >120% |
| | | | | Stage 3 |
| 32 | 30 | 31 | 32 | >120% |
| | | | | Stage 3 |
| 35 | 33 | 34 | 35 | >120% |
| | | | | Stage 3 |
| 38 | 36 | 37 | 38 | >120% |
| | | | | Stage 3 |

### Procedure

Esophageal dilation is a treatment where a balloon catheter apparatus is inserted through the mouth and into the esophagus often around a guide wire. A local anesthesia may numb a patient's throat or a general anesthesia for conscious sedation. Fluoroscopy is used to create a continuous x-ray image on a monitor the distal balloon throughout the procedure. A distal balloon is expanded inside the upper esophagus to stretch muscle fibers inside the upper esophageal sphincter (UES). The balloon catheter is then at least partially deflated and removed.

One embodiment of this disclosure includes a method of manufacturing the balloon dilation catheter assembly 100. The method includes coaxially disposing the inner catheter 140 through the outer catheter 130 and to the distal balloon 120. The inner catheter 140 provides an inner lumen configured to allow a guidewire to be positioned there through as discussed herein. Further, a fluid pathway may be defined between the inner catheter and the outer catheter to selectively provide fluid to the balloon lumen 125.

The proximal hub 110 may be operably coupled to the distal balloon 120 through the outer catheter 130 and the inner catheter 140 such that fluid can be transferred from the proximal hub 110 to the balloon lumen 125 through the fluid pathways between the outer catheter 130 and the inner catheter 140. When the distal balloon 120 is selectively inflated to an inflated state, it may have an at least partially oblong cross section as discussed herein. More specifically, the term “oblong” means that the a cross sectional portion of the distal balloon 120 defined along a plane that is substantially perpendicular to the longitudinal axis 102 has a width that is different from a height of the cross section. For example, referring to FIG. 2D the width Wb is greater than the height (represented by diameter Db). In one example, the distal balloon 120 is formed such that the oblong cross-section is formed in part by a profile of the first balloon 123 on a first side and a profile of the second balloon 124 on a second side.

Part of the manufacturing method may include coupling the radiopaque marker 160 on the outer catheter 130. This may be done using known techniques either before or after the outer catheter 130 is positioned over the inner catheter 140 and the proximal hub 110 is coupled to the distal balloon 120. As discussed herein, the radiopaque marker 160 may be formed of any material that provides visualization markers of the balloon catheter 100 when operably disposed in a patient. In other embodiments contemplated herein, the radiopaque marker 160 may be on the distal balloon 120.

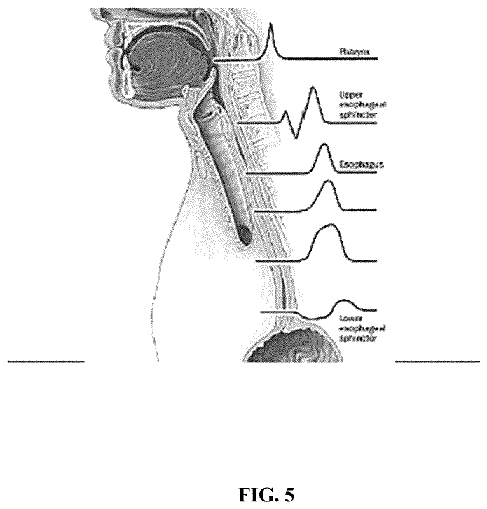

Refer now to FIG. 5 illustrating a cross-sectional view 500 of a patient's upper body, another embodiment of this disclosure is a method of using the catheter assembly 100 on an upper esophageal sphincter 502 of a patient. This method may be enacted by a doctor or other medical professional to address a stricture in a patient's esophagus 504 among other things. This method of use includes providing a substantially deflated distal balloon 120. The distal balloon 120 has an inflated cross-section that is at least partially oblong as discussed herein. The deflated distal balloon 120 is positioned in the upper esophagus 502 area of the patient and then inflated to an inflated state wherein the cross-section is at least partially oblong. A guidewire may be positioned prior to the balloon dilation catheter 100 being inserted along the guidewire. The distal balloon 120 may be inflated by introducing a fluid through the fluid lumen 112 of the proximal hub 110. In one example, the fluid may be a saline solution. However, any fluid capable of expanding the distal balloon 120 may be utilized for this procedure. After the distal balloon 120 has been inflated to dilate and strictures in the upper esophagus 502, the doctor or medical professional may deflated the distal balloon 120 and removing the distal balloon 120 from the upper esophagus 502 area of the patient. Alternatively, the medical professional or doctor may deflate and reposition the distal balloon 120 and execute another inflation procedure before removing the distal balloon 120 from the patient.

In one example of this method, the distal balloon 120 is positioned at least partially within an upper esophageal sphincter of the patient before being inflated. In another part of this method, the distal balloon 120 at least partially dilates the upper esophageal sphincter when the distal balloon 120 is inflated to have the oblong cross-section.

In one example of this disclosure, the distal balloon 120 is formed from a semi-compliant material such as polyamides, and engineered nylons such as Pebax®, Grilamid®, and Vestamid®, PET, and high durometer polyurethane. In another example of this disclosure the burst pressure of the distal balloon 120 is between about 15 psi to about 265 psi.

In another part of this method, the inflating the distal balloon 120 step includes inflating the distal balloon 120 to a first stage of expansion and then inflating the distal balloon to a second stage of expansion. In this configuration, the circumference of the balloon in the second stage of expansion is larger than the circumference of the balloon in the first stage of expansion. Both stages of expansion may be associated with a specific pressure of fluid within the distal balloon 120. As one example of the expansion of the distal balloon 120, the circumference of the distal balloon 120 in the first stage is between about 16 mm and 38 mm. However, any known size needed to expand a stricture in the upper esophagus is considered herein.

Another example of this disclosure is a balloon catheter kit 600. The kit 600 may include a first balloon assembly 602. The first balloon assembly 602 may be substantially the same as the balloon catheter 100 illustrated and described herein. More specifically, the first balloon assembly 602 may have a distal balloon that has an oblong cross-section with a first circumference. The kit may also include a second balloon assembly 604. The second balloon assembly 604 may be substantially the same as the balloon catheter 100 illustrated and described herein with the exception of having different balloon dimensions than the first balloon assembly 602. More specifically, the second balloon assembly 604 may have a distal balloon that has an oblong cross-section with a second circumference that is different from the first balloon assembly 602.

The balloon assemblies 602, 604 may be packaged together in a container 606 that removably contains both the first balloon assembly 602 and the second balloon assembly 604. The container 606 may provide a substantially sterile interior space wherein the balloon assemblies 602, 604 can remain in a sterile environment until the container 606 is opened. In one example, the container may be a sterile paper, plastic, or the like material formed of two elements sealed to one another about the periphery to form the sterile interior space. The two elements may be selectively separated from one another to allow the user to access the balloon assemblies 602, 604 therein when needed.

The balloon catheter kit 600 may also have a third balloon assembly 608. The third balloon assembly 608 may be substantially the same as the balloon catheter 100 illustrated and described herein with the exception of having different balloon dimensions than the first balloon assembly 602 and the second balloon assembly 604. More specifically, the third balloon assembly 608 may have a distal balloon that has an oblong cross-section with a third circumference that is different from the first and second balloon assemblies 602, 604. Further, the third balloon assembly 608 may also be positioned within the sterile interior space of the container 606.

In this balloon catheter kit 600 the second circumference of the second balloon assembly 604 is greater than the first circumference of the first balloon assembly 602. Further, the third circumference of the third balloon assembly 608 is greater than the second circumference. In this configuration, the balloon catheter kit 600 provides a medical professional with balloon assemblies 602, 604, 608 having balloons with a different circumference to be selected as appropriate for the specific procedure and anatomy of the patient. Further still, the balloon assemblies 602, 604, 608 may be used on a patient in sequence to progressively expand a corresponding stricture.

Referring now to Table 2 below, in one aspect of this disclosure each balloon assembly 602, 604, 608 may be a two stage balloon with the following corresponding properties. For example, the first balloon assembly 602 may have a first stage wherein 4 ATM of pressure is introduced to the distal balloon lumen. For the first stage of the first balloon assembly 602 the width of the corresponding balloon may be about 22 mm. For the second stage, 6 ATM may be provided to the balloon lumen to expand the corresponding balloon to a width of about 23 mm. Similarly, the second balloon assembly 604 may have a first stage wherein 2 ATM of pressure are provided to the lumen to expand the balloon to 30 mm wide while the second stage utilizes 4 ATM to expand the balloon to 32 mm side. Further, the third balloon assembly 608 may have a first stage wherein 2 ATM of pressure are provided to the lumen to expand the balloon to 36 mm wide while the second stage utilizes 4 ATM to expand the balloon to 38 mm wide.

While particular pressure and widths are provided herein, this disclosure contemplates others as well. Further, while the corresponding width is discussed associated with the stage and pressure of the balloon, a person skilled in the art understands this alters the corresponding circumference as well. Accordingly, the particular pressures and width are provided as examples while others are also considered herein.

TABLE 2

| | | Pressure | Double Balloon Width |
|---|---|---|---|
| Balloon | Stage | (ATM) | Wb (mm) |
| 602 | 1 | 4 | 22 |
| | 2 | 6 | 23 |
| 604 | 1 | 2 | 30 |
| | 2 | 4 | 32 |
| 608 | 1 | 2 | 36 |
| | 2 | 4 | 38 |

In another aspect of the balloon catheter kit 600, indicators 610 may be positioned on the container 606 to provide specific dimension information about the corresponding balloon assemblies 602, 604, 608. For example, the circumference of the corresponding inflated balloon may be identified by the indicators 610 among other things. Similarly, indicators 612 may be on the balloon assemblies 602, 604, 608 themselves to distinguish the balloon assemblies 602, 604, 608 from one another. The indicators 612 may be color coding that corresponds with the information presented on the container 606 indicators 610. Alternatively, the indicators 612 may be printed information such as serialized numbers or the like. Alternatively, the indicators 612 may present dimension information about that specific balloon assembly. Further, the indicators 612 may be positioned at any location on the balloon assemblies 602, 604, 608.

The balloon catheter kit 600 may also have instructions in or on the container 606 instructing sequential use of the first balloon assembly 602 before the use of the second balloon assembly 604. Further, the instructions may instruct use of the second balloon assembly 604 before use of the third balloon assembly 608. The instructions may be printed on the container 606 as part of the indicators 610 discussed herein. Alternatively, the instructions may be printed on a sterilized paper and packaged in the sterile environment.

In another aspect of this disclosure, a guidewire may be included in the sterile environment as part of the balloon catheter kit 600. The guide wire may be positioned through the balloon dilation catheter 100 and positioned at a desired location of the patient. Then the balloon dilation catheter 100 may be slit along the guide wire until the dilation balloon 120 is properly positioned. In one embodiment, the position may be confirmed with the radiopaque marker. However, any known method is considered herein.

The balloon catheter kit 600 described herein may have any number of balloon assemblies. While the present disclosure illustrates three balloon assemblies 602, 604, 608, this disclosure considers providing only two or only one balloon assembly in the balloon catheter kit 600. Further, this disclosure contemplates providing more than three balloon assemblies within the balloon catheter kit 600. Further, while one example of a container 606 is considered herein, the container 606 may be any configuration that provides a sterilized environment for the balloon assemblies 602, 604, 608.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of manufacturing a balloon dilation catheter assembly, comprising:
coaxially disposing an inner catheter through an outer catheter and to a distal balloon;
operably coupling a proximal hub to the distal balloon through the outer catheter and the inner catheter;
maintaining an inner lumen in the inner catheter configured to pass over a guidewire; and
defining a fluid pathway channel between the inner catheter and the outer catheter by which fluid is selectively directed from the proximal hub to a lumen in the distal balloon;
wherein, the distal balloon is selectively inflatable to an inflated state having an at least partially oblong cross section using the fluid pathway channel.

2. The method of claim 1, further comprising positioning a radiopaque marker on the outer catheter configured for visualization of the balloon catheter when operably disposed in a patient.

3. The method of claim 1, wherein the oblong cross section of the balloon further comprises:
a first balloon profile on a first side; and
a second balloon profile along a second side;
wherein the first balloon profile and second balloon profile are fluidly coupled to one another to form the oblong cross-section of the balloon when in the inflated state.

4. A method of using a catheter for dilating an area of the esophagus, comprising:
providing a substantially deflated balloon having an inflated cross-section that is at least partially oblong;
positioning the balloon in an upper esophagus area of a patient;
inflating the balloon to an inflated state wherein the cross-section is at least partially oblong;
deflating the balloon; and
removing the balloon from the upper esophagus area of the patient.

5. The method of claim 4, wherein the balloon is positioned at least partially within an upper esophageal sphincter of the patient before being inflated.

6. The method of claim 5, wherein the balloon at least partially dilates the upper esophageal sphincter when the balloon is inflated.

7. The method of claim 4, wherein the balloon is comprised of a semi-compliant material selected from the group of polyamides, and engineered nylons such as Pebax®, Grilamid®, and Vestamid®, PET, and high durometer polyurethane.

8. The method of claim 4, wherein the burst pressure of the balloon is between about 15 psi to about 265 psi.

9. The method of claim 4, wherein inflating the balloon further comprises a first stage of expansion and a second stage of expansion.

10. The method of claim 9, wherein the circumference of the balloon in the second stage of expansion is larger than the circumference of the balloon in the first stage of expansion.

11. The method of claim 10, further wherein the width of the balloon in the first stage is not less than 16 mm and not greater than 38 mm.

12. A balloon catheter kit comprising:
a first balloon assembly comprising:
a first balloon having a first lumen fluidly coupled to a first hub to selectively transition the first balloon between a deflated state and an inflated state;
wherein, in the inflated state, the first balloon has an oblong cross-section with a first circumference;
a second balloon assembly comprising:
a second balloon having a second lumen fluidly coupled to a second hub to selectively transition the second balloon between a deflated state and an inflated state;
wherein, in the inflated state, the second balloon has an oblong cross-section with a second circumference;
a container configured to removably contain both the first balloon assembly and the second balloon assembly;
further wherein both the first balloon assembly and the second balloon assembly are sized to pass over a guidewire.

13. The balloon catheter kit of claim 12:
wherein, the first balloon assembly further comprises a first balloon profile on a first side and a second balloon profile along a second side that are fluidly coupled to one another to form the oblong cross-section of the first balloon; and
wherein, the second balloon assembly further comprises a first balloon profile on a first side and a second balloon profile along a second side that are fluidly coupled to one another to form the oblong cross-section of the second balloon.

14. The balloon catheter kit of claim 12, further comprising a third balloon assembly comprising a third balloon having a third lumen fluidly coupled to a third hub to selectively transition the third balloon between a deflated state and an inflated state;
wherein, in the inflated state, the third balloon has an oblong cross-section with a third circumference.

15. The balloon catheter kit of claim 12, wherein the second circumference is greater than the first circumference.

16. The balloon catheter kit of claim 14, wherein the second circumference is greater than the first circumference and the third circumference is greater than the second circumference.

17. The balloon catheter kit of claim 12, further comprising providing indicators on the container providing dimension information for the first balloon assembly and the second balloon assembly.

18. The balloon catheter kit of claim 12, further comprising providing indicators on the first balloon assembly and the second balloon assembly distinguishing the first balloon assembly from the second balloon assembly.

19. The balloon catheter kit of claim 12, further comprising providing instructions in or on the container instructing sequential use of the first balloon assembly before the use of the second balloon assembly.

20. The balloon catheter kit of claim 12, further comprising a guidewire.
